(19) United States
(12) Patent Application Publication
(10) Pub. No.: US 2025/0063311 A1
(43) Pub. Date: Feb. 20, 2025

(54) USER-PREFERRED ADAPTIVE NOISE REDUCTION

(71) Applicant: Cochlear Limited, Macquarie University, NSW (AU)

(72) Inventors: Alexander VON BRASCH, Cremorne, NSW (AU); Stephen FUNG, Dundas Valley, NSW (AU)

(21) Appl. No.: 18/723,578

(22) PCT Filed: Dec. 16, 2022
(86) PCT No.: PCT/IB2022/062392

§ 371 Date: Jun. 24, 2024

Related U.S. Application Data
(60) Provisional application No. 63/294,955, filed on Dec. 30, 2021.

(51) Int. Cl. H04R 25/00; A61N 1/02; A61N 1/36; A61N 1/372; G10L 21/0216

(52) U.S. Cl. CPC H04R 25/43; A61N 1/36038; G10L 21/0216; A61N 1/025; A61N 1/36132; A61N 1/37247; H04R 2225/67; H04R 2460/01

(57) ABSTRACT

Presented herein are techniques for enabling a user of a wearable or implantable device to define noise sources for suppression/attenuation in an ambient environment. In particular, a plurality of devices within the ambient environment form a wearable or implantable system. The plurality of devices capture environmental signals (e.g., sound signals, visual signals, etc.) from the ambient environment and the system determines, from the environmental signals, one or more noise sources present in an ambient environment. The system is configured to determine at least one user-preferred noise source from the one or more noise sources for suppression (attenuation) and, accordingly, suppress the at least one user-preferred noise source within the environmental signals to generate noise-suppressed environmental signals. In certain examples, the system generates stimulation signals from the noise-suppressed environmental signals and the system delivers the stimulation signals to a user.

### BACKGROUND

### Field of the Invention

The present invention relates generally to adaptive noise reduction in wearable or implantable systems.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: capturing sound signals at a hearing device configured to be worn by a user and at one or more remote devices in wireless communication with the hearing device; determining, based on the sound signals, one or more noises present in an ambient environment of the hearing device; determining at least one user-preferred noise from the one or more noises for suppression; and suppressing the at least one user-preferred noise within the sound signals to generate noise-suppressed sound signals.

In another aspect, one or more non-transitory computer readable storage media are provided. The one or more non-transitory computer readable storage media comprise instructions that, when executed by a processor of a hearing device, cause the processor to: receive, at the hearing device configured to be worn by a user, noise model parameters from at least one external device in wireless communication with the hearing device, wherein the noise model parameters represent noise detected by the at least one external device; determine, based on sound signals received at the hearing device and the noise model parameters, one or more noises present in an ambient environment of the hearing device; determine at least one user-preferred noise from the one or more noises for suppression; suppress the at least one user-preferred noise within the sound signals to generate noise-suppressed sound signals; and process the noise-suppressed sound signals for generation of stimulation signals for delivery to the user.

In another aspect, a method is provided. The method comprises: capturing environmental signals at an implantable medical device system; determining, based on the environmental signals, one or more noises present in an ambient environment of a user of the implantable medical device system; determining at least one user-preferred noise from the one or more noises; attenuating the at least one user-preferred noise within the environmental signals to generate noise-reduced environmental signals; and generating, based on the noise-reduced environmental signals, one or more stimulation signals for delivery to the user of the implantable medical device system.

In another aspect, a system is provided. The system comprises: a user device is configured to be worn by a user comprising one or more sensors configured to capture environmental signals; one or more remote devices in wireless communication with the user device, wherein the one or more remote devices each include at least one sensor configured to capture environmental signals; and one or more processors configured to: determine, based on the environmental signals, one or more noises present in an ambient environment of the user device, determine at least one user-preferred noise from the one or more noises for suppression, and suppress the at least one user-preferred noise within the environmental signals to generate noise-suppressed environmental signals.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a schematic diagram illustrating aspects of the techniques presented herein;

FIG. 3A is a functional block diagram illustrating a user-preferred noise cancellation system, in accordance with certain embodiments presented herein;

FIG. 3B is a functional block diagram illustrating one implementation of a user preference module of FIG. 3A;

FIGS. 4A and 4B are diagrams schematically illustrating generation of a noise model in accordance with embodiments presented herein;

FIGS. 5A, 5B, 5C, and 5D are a series of diagrams schematically illustrating example noise suppression operations, in accordance with certain embodiments presented herein;

FIGS. 6A, 6B, 6C, 6D, and 6E are a series of diagrams illustrating simplified user interfaces, in accordance with certain embodiments presented herein;

FIG. 7 is a functional block diagram illustrating training and final operation of a noise suppression prioritization module to automatically select a user-preferred noise for suppression/attenuation, in accordance with embodiments presented herein;

FIG. 8 is a flowchart of an example method, in accordance with certain embodiments presented herein;

FIG. 9 is a schematic diagram illustrating a retinal prosthesis system with which aspects of the techniques presented herein can be implemented; and

FIG. 10 is a flowchart of another example method, in accordance with certain embodiments presented herein.

### DETAILED DESCRIPTION

Presented herein are techniques for enabling a user of a wearable or implantable device to define noise sources for suppression/attenuation in an ambient environment. In particular, a plurality of devices within the ambient environment form a wearable or implantable system. The plurality of devices capture environmental signals (e.g., sound signals, light signals, etc.) from the ambient environment and the system determines, from the environmental signals, one or more noises (e.g., noise sources, noise types, etc.) present in an ambient environment. The system is configured to determine at least one user-preferred noise from the one or more noises for suppression (attenuation) and, accordingly, suppress the at least one user-preferred noise within the environmental signals to generate noise-suppressed environmental signals. In certain examples, the system generates stimulation signals from the noise-suppressed environmental signals and the system delivers the stimulation signals to a user.

More specifically, hearing and other types of devices can only do so much with their limited inputs (e.g., the limitation of processing power and memory usage) to eliminate the background noise mixed with the target signal (signal of interest). Generally, it is a one-size-fit-all approach to cancel/suppress the noise in the background. However, presented herein are techniques that make use of the presence of multiple microphones provided by network-connected devices in order to provide an improved noise reduction/suppression system. In particular, and as described further below, the techniques presented herein create a profile showing the existing types of background noise (noise) in the ambient environment, estimate/build a likelihood metric system, learn to prioritize mitigating different noise types depending on the user's preference, and pass the noise parameters from the analysis model to the hearing device for use in its noise cancellation algorithm.

The proposed system is an adaptive system that can identify, prioritize, and suppress the background noise(s) which are relevant to that specific user. Besides reducing the background noise(s) in the general term, the proposed system can adaptively prioritize, suppress, and update the model for real time noise reduction to reduce the noise(s) that are relevant to the user. In certain aspects, user input is used to select which components of the background noise should be attenuated, with a learning aspect proposed.

Merely for ease of description, the techniques presented herein are primarily described with reference to a specific implantable medical device system, namely a cochlear implant system, and with reference to a specific type of environmental signals, namely sound signals. However, it is to be appreciated that the techniques presented herein may also be partially or fully implemented by other types of devices or systems with other types of environmental signals. For example, the techniques presented herein may be implemented by other hearing devices, personal sound amplification products (PSAPs), or hearing device systems that include one or more other types of hearing devices, such as hearing aids, middle ear auditory prostheses, bone conduction devices, direct acoustic stimulators, electro-acoustic prostheses, auditory brain stimulators, cochlear implants, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, wearable devices, etc.

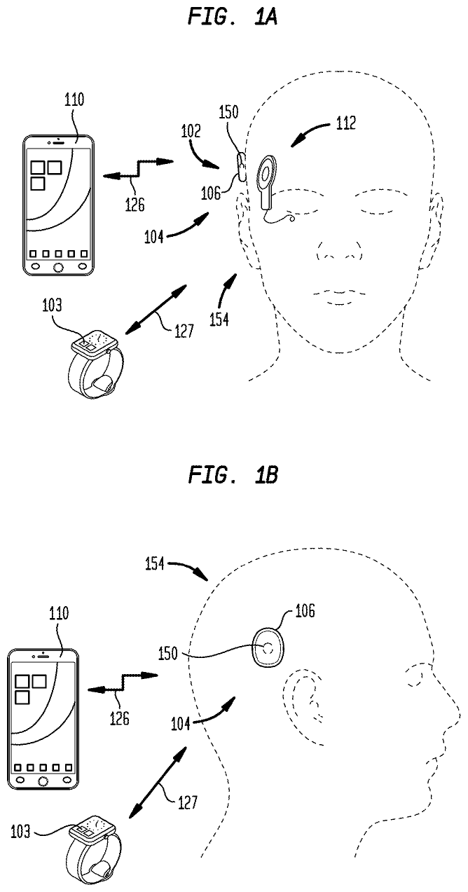

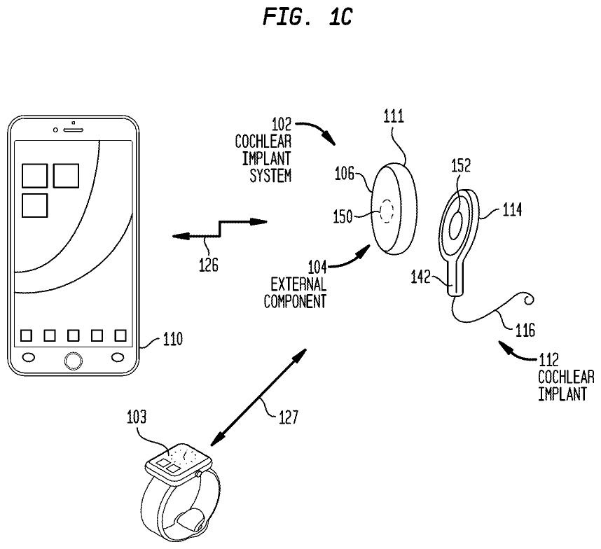

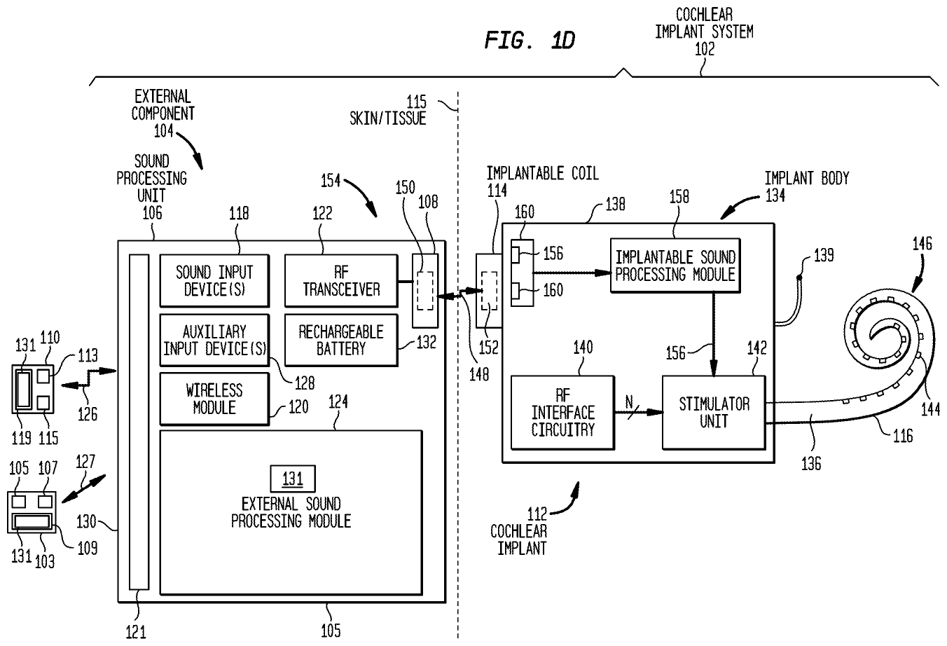

FIGS. 1A-1D illustrates an example cochlear implant system 102 with which aspects of the techniques presented herein can be implemented. The cochlear implant system 102 comprises an external component 104, an implantable component 112, an external device 110, and a remote sensor device 103 which, in this example, is a wearable device 103. In the examples of FIGS. 1A-ID, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

As noted, cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external component that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

Also shown in FIGS. 1A-1D is a remote sensor device in the form of a wearable device 103. In this example, the wearable device 103 comprises at least one microphone 105, a wireless module (e.g., transmitter, receiver, and/or transceiver) 107 (e.g., for communication with the external device 110 and/or the sound processing unit 106), and a processing module 109 comprising user-preferred noise suppression logic 131. It is to be appreciated that use of a wearable device 103 comprising at least one microphone 105 is merely illustrative and that the remote sensor device may include alternative types of input devices.

The processing module 109 may comprise, for example, one or more processors, and a memory device (memory) that includes the user-preferred noise suppression logic 131. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the user-preferred noise suppression logic 131 stored in the memory device.

The wearable device 103 and the sound processing unit 106 (or the cochlear implant 112) wirelessly communicate via a communication link 127. The communication link 127 may comprise, for example, a short-range communication link, such as Bluetooth link, Bluetooth Low Energy (BLE) link, a proprietary link, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 comprises the external device 110 and the wearable device 103. The external device 110 is a computing device, such as a computer (e.g., laptop, desktop, tablet), a mobile phone, remote control unit, etc. In this example, the external device 110 comprises at least one microphone 113, a wireless module (e.g., transmitter, receiver, and/or transceiver) 115 (e.g., for communication with the wearable device 103 and/or the sound processing unit 106), and a processing module 119 comprising user-preferred noise suppression logic 131. It is to be appreciated that external device 110 rising at least one microphone 113 is merely illustrative and that the external device 110 may include alternative types of input sensors.

The processing module 119 may comprise, for example, one or more processors, and a memory device (memory) that includes the user-preferred noise suppression logic 131. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the user-preferred noise suppression logic 131 stored in the memory device.

The external device 110 and the sound processing unit 106 (or the cochlear implant 112) wirelessly communicate via a communication link 126. The communication link 126 may comprise, for example, a short-range communication link, such as Bluetooth link, Bluetooth Low Energy (BLE) link, a proprietary link, etc.

The OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless module (e.g., transmitter, receiver, and/or transceiver) 120 (e.g., for communication with the external device 110). However, it is to be appreciated that one or more input devices may include additional types of input devices and/or less input devices (e.g., the wireless module 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 may comprise, for example, one or more processors, and a memory device (memory) that includes user-preferred noise suppression logic 131.

The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the user-preferred noise suppression logic 131 stored in the memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient. The input signals can comprise signals received at the external component 104 (e.g., received at sound input devices 118), signals received at the wearable device 103, and/or signals received at the external device 110.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

Different technologies exist to extract the vital information out of the sound signals for processing the speech information to help a user better perceives the audio in the ambient environment. In conventional systems, hearing devices are only able to process based on the sound signals received at its own microphones. In general, a hearing device can only do so much based on its limited inputs (e.g., the limitation of processing power and memory usage) to eliminate the background noise mixed with the signal of interest.

With the growth of the Internet of Things and wireless connectivity between electronic devices (e.g., the Bluetooth Low Energy), devices can communicate with each other within a network (e.g., body area network). Increasingly, these devices include microphones or other input sensors that can capture information about the ambient environment of a hearing device. For instance, it is increasingly common for mobile phones, wearable devices, to include one or more input sensors (e.g., microphones). As such, presented herein are techniques that leverage the input sensors of other devices in a process that determines the presence and type of background noise around the hearing device. Depending on the determined type of noise, the additional information contributed by the other supporting device(s) can be used to, for example, construct an adaptive masking scheme to filter out user-preferred noise.

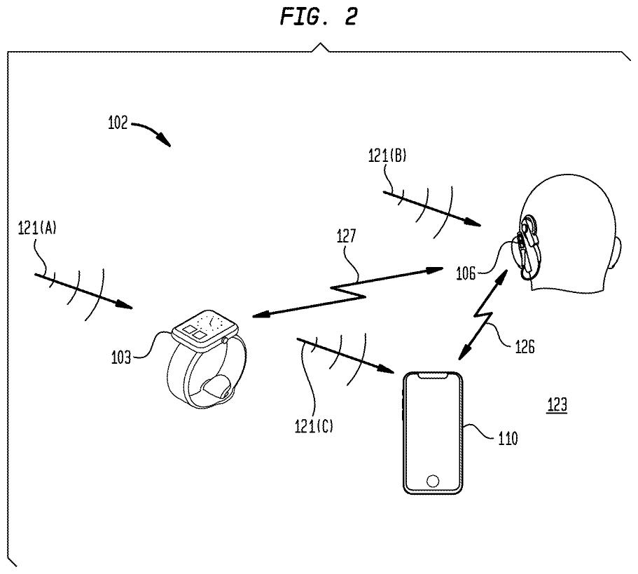

FIG. 2 is a schematic diagram illustrating aspects of the techniques presented herein. For ease of illustration, FIG. 2 is described with reference to cochlear implant system 102 described above with reference to FIGS. 1A-1D. In particular, shown in FIG. 2 is the sound processing unit 106, the wearable device 103, and the external device 110.

In general, FIG. 2 illustrates a system that is able to utilize the microphones of the connecting devices, namely the microphone(s) 105 of wearable device 103, and the microphone(s) 113 of the external device 110, in a body area network to generate a real-time environmental profile based on the continuous changing background noise (static and/or dynamic) existing in the environment and use of this real-time profile to determine at least one user-preferred noise source for suppression (attenuation).

More specifically, as shown in FIG. 2, the wearable device 103, the sound processing unit 106, and the external device 110 receive sound signals 121(A), 121(B), and 121(C), respectively, from the ambient environment 123 in which the cochlear implant system 102 is positioned/located. The sound signals 121(A), 121(B), and 121(C) are captured from the same ambient environment 123 and, as such, generally include the same sound sources. However, the different locations of the wearable device 103, the sound processing unit 106, and the external device 110 capture those same sounds differently, meaning that certain attributes of the sound sources in the ambient environment 123 will be different in each of the sound signals 121(A), 121(B), and 121(C). The techniques presented herein leverage the differences between the sound signals 121(A), 121(B), and 121(C) in order to determine one or more noises (e.g., noise sources, noise types, etc.) present in the ambient environment 123. The techniques presented herein further determine at least one user-preferred noise from the one or more noises for suppression (attenuation).

More specifically, presented herein is a user-preferred noise suppression system that is configured to use environmental signals, such as sound signals, captured from multiple sources (e.g., the sound signals 121(A), 121(B), and 121(C)) to generate a profile of the noise present in the ambient environment (e.g., representing the nature/attributes of the detected noise in the ambient environment 123). The user-preferred noise suppression system is configured to determine at least one user-preferred noise for suppression.

In certain embodiments, the system can classify/categorize the noise into different “noise categories.” The noise categories can be, for example different type of noise, different noise sources, different shared sound attributes (e.g., high frequency, low frequency, etc.). The system can allow a user to select, in real-time, specific noises, or noise categories, for suppression/attenuation (e.g., cancellation). In certain embodiments, the system is configured to learn and automatically feedback some particular data to an adaptive masking system to suppress or cancel certain user-preferred noises (e.g., noise patterns, etc.).

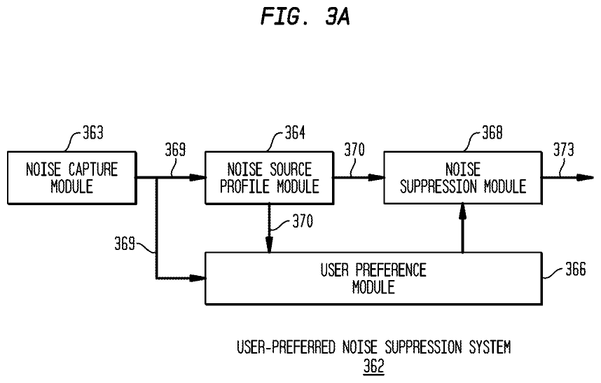

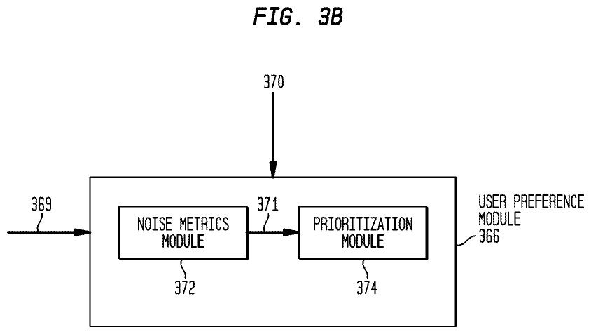

FIG. 3A is a functional representation of a user-preferred noise suppression system in accordance with certain embodiments presented herein, referred to as user-preferred noise suppression system 362. For ease of description, the user-preferred noise suppression system 362 is described with reference to cochlear implant system 102 of FIGS. 1A-1D and FIG. 2. In general, the various operations of the user-preferred noise suppression system 362, as shown in FIG. 3A, are enabled by the user-preferred noise suppression logic 131 (FIG. 1D). That is, the user-preferred noise suppression logic 131 represents one example implementation of certain elements of the user-preferred noise suppression system 362 shown in FIG. 3A, where the different operations/modules could be performed at different physical devices.

Turning to the example of FIG. 3A, the user-preferred noise suppression system 362 is formed by a number of functional modules that include a noise capture module 363, a noise source profile module 364, a user preference module 366, and a noise suppression module 368.

As noted, these modules 363, 364, 366, and 368 can each be implemented by different components of a wearable or implantable system. For example, certain modules can be implemented by components of a wearable device (e.g., sound processing unit 106 in FIGS. 1A-1D and FIG. 2), an implantable device (e.g., cochlear implant 112 in FIGS. 1A-1D and FIG. 2), or an auxiliary devices (e.g., external device 110 or wearable device 103 in in FIGS. 1A-1D and FIG. 2).

Returning to the example of FIG. 3A, the noise capture module 363 is configured to capture and monitor the background noise in the ambient environment 123. The noise capture module 363 can comprise, for example, the microphones/sound input devices of the various devices that capture sound signals from the ambient environment, such as the microphones/sound input devices of sound processing unit 106, external device 110, and/or wearable device 103 in in FIGS. 1A-1D and FIG. 2.

The noise source profile module 364 is configured to create a “noise model” or “noise profile” showing the existing types/sources of background noise in the ambient environment. For instance, the noise model can include the fundamental and/or harmonics of the noises, approximate frequency range of the noise, repeatability/periodicity of the noise, the amplitude/duration of the noise, etc. In general, the noise models are parameters that describe the noise so the entire signal captured by the external device 110 and or remote device 103 are not streamed to the sound processing unit 106. The parameters could be filter coefficients for use by the sound processing unit 106.

The user preference module 366 is configured to determine, using the sound signals, the noise profile, and/or other information, at least one user-preferred noise, which is present in the ambient environment 123, for suppression/attenuation. That is, the user preference module 366 is configured to determine which of the noises (e.g., noises sources, noise types, noise attributes, etc.) present in the sound signals 121(A)-121(C) are preferred, by the user, for suppression. The user preference module 366 could be implemented, for example, at the external device 110, remote device 103, and/or the sound processing unit 106.

In certain embodiments, the user preference module 366 is configured to determine the user-preferred noise source for suppression based on a user input. For example, the system 102 (e.g., external device 110) can be configured to provide the user with an indication of the one or more noises present in an ambient environment 123 (e.g., determined from the noise profile module 364). The system can then receive (e.g., from the user, a caregiver, etc.) a selection of the at least one user-preferred noise to suppress (e.g., a user input identifying one of the noise categories for suppression).

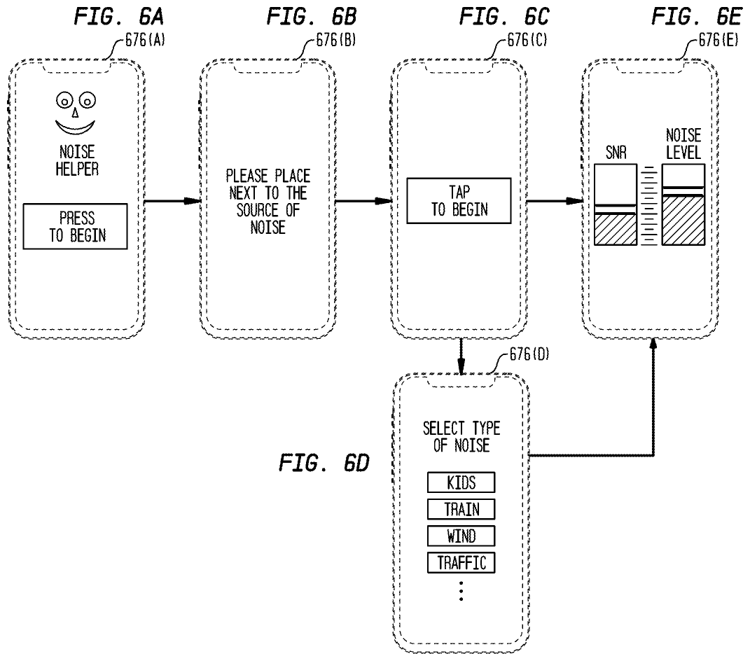

In one such example, the system provides the recipient a list of determined noise categories (e.g., as shown below in FIG. 6D). For example, the system is configured to display, at a display screen of the external device 110, a list of the one or more noises present in an ambient environment and the user, caregiver, etc. enters an input to select one of the one or more noises (e.g. noise categories) present in the ambient environment for suppression.

In other embodiments, the user preference module 366 is configured to automatically determine the user-preferred noise source for suppression based on a user input. In such embodiments, the user preference module 366 can be implemented as, for example, a machine-learning system. In such embodiments, the machine-learning system is configured to determine which of the one or more noise sources should be suppressed to provide the user with an optimal listening experience. This determination can be made based on a number of different factors, but is generally based on machine-learning preferences of the user and attributes of the sound signals themselves.

In certain embodiments, the selection of noises for cancellation by the user can form part of a training process for the machine-learning system. That is, in certain embodiments, the system initially relies on user inputs to determine which noises to suppress. Over time, the system can use machine-learning to progress to, for example, providing the user with a recommendation of a noise to suppress and, eventually, automatically selecting a noise to suppress. The user can also selectively activate/deactivate the user-preferred noise suppression system 362, override a selection made by the user-preferred noise suppression system 362, etc.

FIG. 3B illustrates one example of a machine-learning system configured to user-preferred noise source for suppression. In this example, the user preference module 366 comprises a noise metrics module 372 that is configured to estimate and build a likelihood metric 371 that the type(s) of noise would continue to exist in the ambient background (e.g. utilizing geographical data, or stochastic modelling of the recorded noise). For example, the background noise can be static, dynamic, or both. The likelihood determination determines the likelihood that a signal is noise and likelihood it will continue.

Also shown is a noise suppression prioritization module 374 that is configured to learn to prioritize suppression of different noises (e.g., different noise types), by incorporating the attributes of the noise, likelihood metric 371, and other factors. That is, the noise suppression prioritization module 374 is a machine-learning algorithm that is configured to learn the attributes of noise types that the user prefers to suppress. For example, the suppression prioritization module 374 can learn to prioritize suppression of different noises based on the physiological and/or cognitive state of that user or objective measures (e.g., electrically evoked compound action potential (ECAP) measurements, electrocochleography (ECoG) measurements, higher evoked potentials measured from the brainstem and auditory cortex, and measurements of the electrical properties of the cochlea and the electrode array, electrophysiological measurements, etc.), etc. In certain embodiments, the noise suppression prioritization module 374 is configured to learn the attributes of noise types that the user prefers to suppress through audio processing mechanisms (e.g., learn some common characteristics shared between the majority of noise types, such as low frequency, impulsive, continuous or intermittent, etc.). In certain embodiments, the noise suppression prioritization module 374 is configured to learn the attributes of noise types that the user prefers to suppress through subjective measures. Subjective measures can be considered, for example, relative to that particular individual (there could be a machine learning model driven behind the scene to learn the reactions of that particular individual when he/she is exposed to different types of sounds and any sounds resulting in having the individual responding in an unpleasant manner could be considered noise ‘i.e. the unwanted sound’). In another example, the subjective measurements can be based a larger portion of the population (e.g., if over 70% of users would respond to a given sound in a negative way, that sound source could be considered a noise source).

Regardless of the user preference module 366 implementation (e.g., manual selection, recommendation, or automated selection), an indication of the selected at least one user-preferred noise to suppress can be provided to the noise suppression module 368. The noise suppression module 368 uses this information to generate noise-suppressed sound signals 373 (e.g., signals in which the least one user-preferred noise source has been cancelled, reduced, attenuated, or otherwise suppressed).

With specific reference to the example of FIG. 2, the user-preferred noise suppression system 362 can include the sound processing unit 106, the external device (e.g., Smart Phone) 110, and the wearable device 103. The external device 110 and the wearable device 103 are each paired with the sound processing unit 106 in a body area network, which is represented by connections 126 and 127. Although not shown in FIGS. 1A-1D and FIG. 2, the external device 110 and the wearable device 103 can also, in certain embodiments, communicate with one another via a wireless connection (e.g., the body area network includes a communication link between the external device 110 and the wearable device 103). Moreover, it is to be appreciated that the external device 110 and the wearable device 103 are merely illustrative and that other devices could also or alternatively be present in the body area network.

As noted, the wearable device 103 comprises at least one microphone 105 that is configured to capture sound signals 121 (A) from the ambient environment 123. Similarly, the external device 110 comprises at least one microphone 113 configured to capture sound signals 121(C) from the ambient environment 123. In certain examples, the microphones 105, 113, as well as the microphones 118 of the sound processing unit 106, form the noise capture module 363 of FIG. 3A. In FIG. 3A, the sound signals 121(A), 121(B), and 121(C) are represented by arrow 369.

The wearable device 103 and the external device 110 are configured to process the respective sound signals 121(A) and 121(C) received thereby and, in certain embodiments, are configured to construct the noise model (noise profile) of the noise present in the ambient environment 123. In certain examples, the sound processing unit 106 is also configured to generate a noise model from the sound signals 121 (B) received at the microphones 118. That is, in certain embodiments, the wearable device 103, the external device 110, and the sound processing unit 106 each implement aspects of the noise source profile module 364, described above. Since, as noted, the sound signals 121(A), 121(B), and 121(C) have different attributes of the noise and other sound sources present in the ambient environment, the noise models generated by the wearable device 103, the external device 110, and the sound processing unit 106 may differ in certain respects. In FIG. 3A, the noise models are represented by arrow 370.

The user-preferred noise suppression system 362 is advantageous in that it is not a one-size-fit-all approach. Instead, it is an adaptive system to apply the customized noise masking scheme. In particular, from the system's perspective, a signal may be classified as noise signal, but different user can have different levels of acceptance and/or influencing factors and, as such, their acceptance or problems with the same type of noise could be different. In certain embodiments, the proposed system would also take into the account of the individual's level of acceptance when prioritizing the extent of the types of noise showing up in the user-specific profile. For instance, for this person, they may be more sensitive to this type of noise than other types of noises. He/she would try to turn away upon hearing such noise. On the other hand, a person may not be able to hear a particular frequency because of the medical conditions, aging, noise exposure, etc. Thus, if the background noise happens to occur at this frequency and/or range, the system may prioritize this noise to the bottom of the list (after having matched with the user's body condition) freeing up the system resources to handle other dominant background noises.

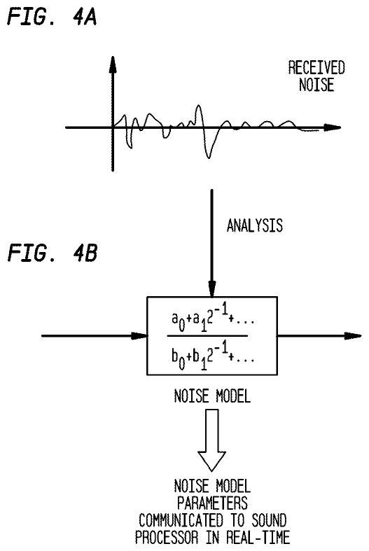

FIGS. 4A and 4B schematically illustrate generation of a noise model in accordance with embodiments presented herein. More specifically, FIG. 4A is a graph representing the noise received by, for example, the external device 110 and FIG. 4B illustrates the corresponding noise model generated from the received noise of FIG. 4A. That is, FIG. 4B illustrates how the external device 110 determines the parameters of a model of the surrounding noise environment (e.g., transfer function plus excitation vector).

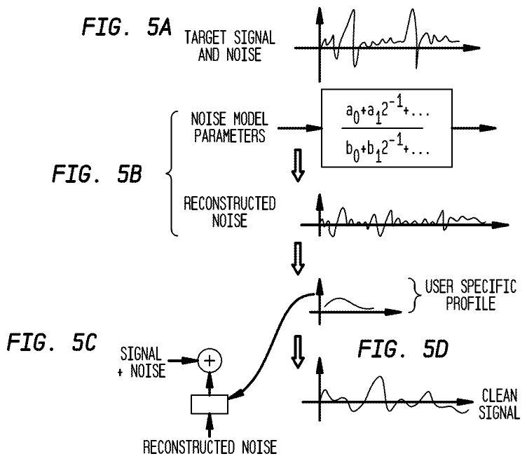

In certain examples, the wearable device 103 and the external device 110 send, in real-time, the parameters of their respective noise model to the sound processing unit 106, which can then use these noise models (potentially with its own noise model) to reduce the incident input noise via noise cancellation/suppression techniques. An example of this noise cancellation could be an Active Noise Cancellation (ANC) system, where the noise model parameters are used to regenerate the noise signal in the sound processing unit 102, and this is then used to subtract from the input signal to reduce the noise component, using standard ANC techniques (such as a Kalman filter). FIGS. 5A, 5B, 5C, and 5D schematically illustrate example operations performed at the sound processing unit 106, in one example implementation.

More specifically, FIG. 5A is graph illustrating the sound signals 121(C) received by the sound processing unit 106. As shown, the sound signals 121(C) comprise both target/desired signals and noise. As shown in FIG. 5B, using the noise model parameters received from the wearable device 103 and/or the external device 110, the sound processing unit 106 reconstructs the noise detected by wearable device 103 and the external device 110. That is, the sound processing unit 106 uses the parameters from the wearable device 103, the external device 110, and/or its own analysis, to determine the attributes of the noise, as detected by the different devices in the body area network, present in the ambient environment 103.

As noted, the techniques presented herein enable the suppression of user-preferred noise (e.g., noises sources, types, etc.) present in the ambient environment 123. As such, as shown in FIG. 5C, the sound processing unit 106 applies a user-specific profile, representing the user-preferred noise sources/types present in the ambient environment, to the reconstructed noise (e.g., filters the reconstructed noise based on a user-specific profile), and then subtracts the filtered-reconstructed noise from the sound signal 121(C) to generate a noise-suppressed/noise-reduced signal. The noise reduced signal is shown in FIG. 5D.

As noted above, in certain examples, the sound processing unit 106 is configured to provide active noise cancellation of user-preferred noises present in the ambient environment 123. Active noise cancellation is based on the presence of at least two input signals, where one input signal is considered to be include predominantly noise and the other signal(s) is considered to include both target signal and noise (target signal +noise). In a simple form, active noise cancellation generates a noise-reduced output by summing together the target signal +noise input and an inverted version of the noise input. In practice, an adaptive algorithm, like a Kalman filter, is used to determine the output (filter the noise to subtract from the input to better handle variations in levels and frequencies, etc.). In the present application, the input microphone(s) on the sound processing unit 106 receive the signal and noise, while the microphones of the remote device 103 and/or external device 110 receive predominantly noise, and so can then be used to subtract the noise from the input.

FIGS. 6A-6E are a series of diagrams illustrating simplified user interfaces for use of the techniques presented herein in active noise cancellation, in accordance with certain embodiments. More specifically, FIG. 6A illustrates an example user interface 676 (A) to active the user-preferred noise suppression techniques presented herein. The user interface 676(A) is displayed, for example, at the external device 110 of FIGS. 1A-1D and 2. FIG. 6B illustrates an example user interface 676(B) instructing the user to place the external device 110 next to a source of noise in the ambient environment 123. In alternative embodiments, the user interface 676(B) could instead instruct the user to “point the phone in the direction of the noise source” or provide another instruction. In still other embodiments, the user interface 676(B) could be omitted.

FIG. 6C illustrates an example user interface 676(C) allowing the user to initiate the user-preferred noise suppression. FIG. 6D illustrates an example user interface 676(D) that displays the noise present in the ambient environment 123. More specifically, FIG. 6D represents an example displayed list of categories of noise sources present in the ambient environment 123. The user, caregiver, etc. can enter an input at user interface 676(D) to select one of the one or more noise sources present in an ambient environment 123 for suppression. FIG. 6E illustrates an optional user interface 676(E) for advanced users that can display sound metrics (e.g., SNR, noise level, etc.). In the examples of FIGS. 6A-6E, the list of noise sources that could be removed would not be fixed, but determined in real-time based on the user-preferences and the ambient environment.

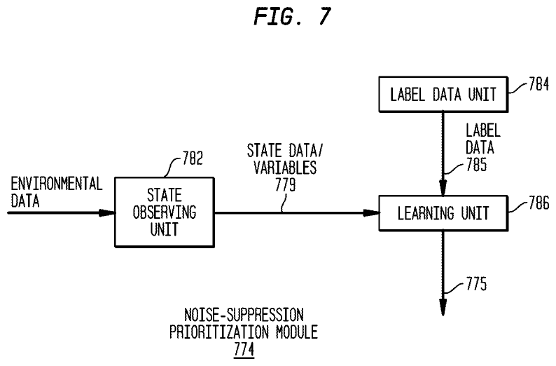

As noted above, certain aspects presented herein use a machine-learning device, referred to as a noise suppression prioritization module, to determine which noises should be suppressed in the ambient environment of a user (e.g., identifying the noises that are preferred by the user for suppression in the environment and proactively suppressing those noises). The noise suppression prioritization module is a functional block (e.g., one or more processors operating based on code, algorithm(s), etc.) that is trained, through a machine-learning process, to select a noise for suppression, while accounting for the user's preferences and attributes of the ambient environment. FIG. 7 is a functional block diagram illustrating training and final operation of a machine-learning device, referred to as noise suppression prioritization module 774, to automatically select a user-preferred noise for suppression/attenuation in accordance with embodiments presented herein.

As shown, the noise suppression prioritization module 774 includes a state observing unit (state unit) 782, a label data unit 784, and a learning unit 786. As described below, the noise suppression prioritization module 774 is configured to generate data 775 presenting the user-preferred noise for suppressed. Stated differently, the noise suppression prioritization module 774 is configured to determine noise source present in the ambient environment that, according to the user's preferences, should be suppressed.

In the example of FIG. 7, the learning unit 786 receives inputs from the state observing unit 782 and the label data unit 784 in order to learn to select a noise source for suppression that accounts for the user's preferences and attributes of the ambient environment. In particular, the state observing unit 782 provides state data/variables, represented by arrow 779, to the learning unit 786. The state data 779 includes data representing the current ambient environment of the user, such as the current sound environment of the user, current light environment of the user, etc. In certain examples, the state data 779 could also include physiological data, which is data representing the current physiological state of the user. This physiological data can include data representing, for example, heart rate, heart rate variability, skin conductance, neural activity, etc. The physiological data can also include data representing the current stress state of the user.

In general, the preferred noise source for suppression is subjective for the user and does not follow a linear function corresponding to the state data 779. That is, the user-preferred noise source for suppression cannot be predicted for different users based on the state data. Therefore, the label data unit 784 also provides the learning unit 786 with label data, represented by arrow 785, to collect the subjective experience/preferences of the user, which is highly user specific. Stated differently, the label data unit 784 collects subjective user inputs of the user's preferred noise sources for cancellation, which is represented in the label data 785. Through machine-learning techniques, the learning unit 786 correlates the state data 779 and the label data 785, over time, to develop the ability to automatically select a user preferred noise source for suppression, user, given the specific attributes of the ambient environment and the user's subjective preferences. Stated differently, the learning unit 786 develops the ability to identify the noises that are preferred by the user for suppression in the environment and proactively suppressing those noises. As a result, the noise suppression is specifically tailored to the noise attributes that are most problematic for the specific user.

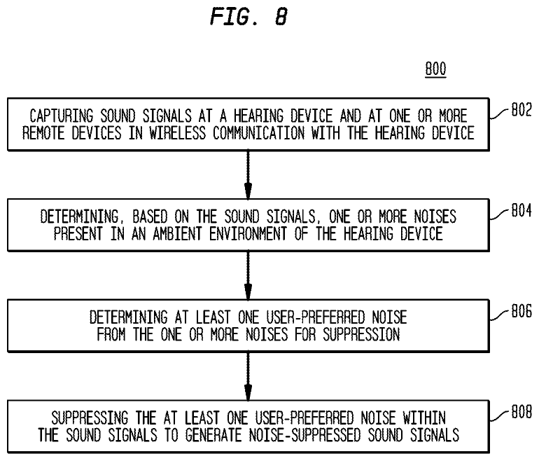

FIG. 8 is flowchart of an example method 800 performed at a hearing device system comprising a hearing device and at one or more remote devices, in accordance with certain embodiments presented herein. Method 800 begins at 802 where sound signals are captured at a hearing device and at one or more remote devices in wireless communication with the hearing device. At 804, the hearing device system determines, based on the sound signals, one or more noises present in an ambient environment of the hearing device. At 806, the device system determines at least one user-preferred noise from the one or more noises for suppression. At 808, the device system determines suppresses the at least one user-preferred noise within the sound signals to generate noise-suppressed sound signals.

As previously described, the techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. For example, besides hearing, the techniques presented herein could be used by retinal prostheses where the “noise” refers to the content of visible signals (e.g., color level, brightness, etc.), rather than sound signals. That is, in these examples, the ‘noise’ would be related to the content of the light (for example) where different vision impaired users may be sensitive to different kinds of light.

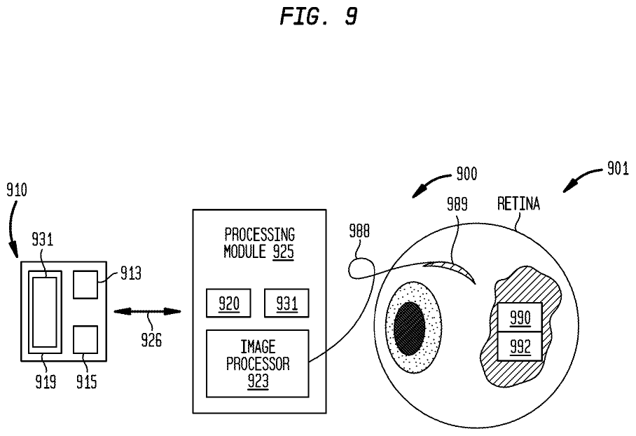

FIG. 9 illustrates a retinal prosthesis system 901 that comprises an external device 910 (which can correspond to the external device 110) configured to communicate with a retinal prosthesis 900. More specifically, the external device 910 is a computing device, such as a computer (e.g., laptop, desktop, tablet), a mobile phone, remote control unit, etc. In this example, the external device 910 comprises at least one light sensor 913, a wireless module (e.g., transmitter, receiver, and/or transceiver) 915 (e.g., for communication with the retinal prosthesis 900), and a processing module 919 comprising user-preferred noise suppression logic 931. It is to be appreciated that external device 910 rising at least one light sensor 913 is merely illustrative and that the external device 110 may include alternative types of input sensors.

The processing module 919 may comprise, for example, one or more processors, and a memory device (memory) that includes the user-preferred noise suppression logic 931. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the user-preferred noise suppression logic 931 stored in the memory device.

The external device 910 and the retinal prosthesis 900 wirelessly communicate via a communication link 926. The communication link 926 may comprise, for example, a short-range communication link, such as Bluetooth link, Bluetooth Low Energy (BLE) link, a proprietary link, etc.

The retinal prosthesis 900 comprises an implanted processing module 925 and a retinal prosthesis sensor-stimulator 990 is positioned proximate the retina of a recipient. In an example, sensory inputs (e.g., photons entering the eye) are absorbed by a microelectronic array of the sensor-stimulator 990 that is hybridized to a glass piece 992 including, for example, an embedded array of microwires. The glass can have a curved surface that conforms to the inner radius of the retina. The sensor-stimulator 990 can include a microelectronic imaging device that can be made of thin silicon containing integrated circuitry that converts the incident photons to an electronic charge.

The processing module 925 includes a wireless module 920, user-preferred noise suppression logic 931, and an image processor 923 that is in signal communication with the sensor-stimulator 990 via, for example, a lead 988 which extends through surgical incision 989 formed in the eye wall. In other examples, processing module 925 is in wireless communication with the sensor-stimulator 990. The image processor 923 processes the input into the sensor-stimulator 990, and provides control signals back to the sensor-stimulator 990 so the device can provide an output to the optic nerve. That said, in an alternate example, the processing is executed by a component proximate to, or integrated with, the sensor-stimulator 990. The electric charge resulting from the conversion of the incident photons is converted to a proportional amount of electronic current which is input to a nearby retinal cell layer. The cells fire and a signal is sent to the optic nerve, thus inducing a sight perception.

The processing module 925 can be implanted in the recipient and function by communicating with the external device 910, such as a behind-the-ear unit, a pair of eyeglasses, etc. The external device 910 can include an external light/image capture device (e.g., located in/on a behind-the-ear device or a pair of glasses, etc.), while, as noted above, in some examples, the sensor-stimulator 990 captures light/images, which sensor-stimulator is implanted in the recipient.

As noted, the external device 910 and the retinal prosthesis 900 include user-preferred noise suppression logic 931. Similar to the above embodiments, the user-preferred noise suppression logic 931 represents a user-preferred noise suppression system that is configured to use light signals captured from multiple sources (e.g., by external device 910 and retinal prosthesis 900 to generate a profile of the light noise sources present in the ambient environment. Using the profile, the user-preferred noise suppression system can determine the nature of the detected background noise(s) in the ambient environment. The user-preferred noise suppression system can then, for example, allow a user to select specific noise sources for suppression or cancellation, learn and automatically feedback some particular data to adaptive masking system to suppress or cancel certain noise patterns, etc. (e.g., filter out user-preferred light noise).

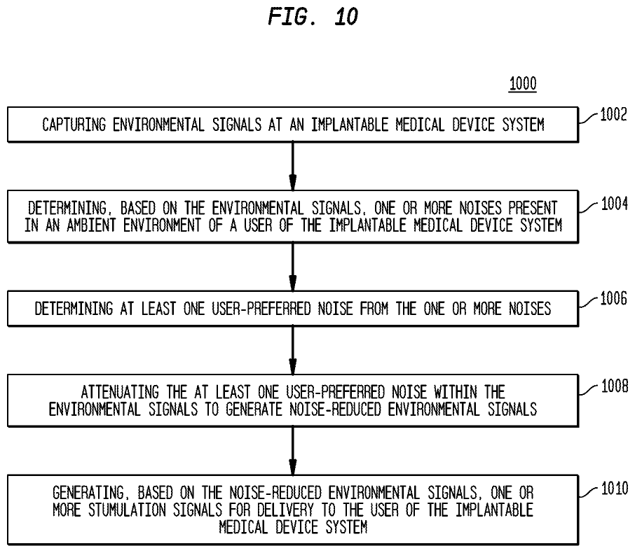

FIG. 10 is flowchart of an example method 1000 performed at an implantable medical device system. Method 1000 begins at 1002 where the implantable medical device system captures environmental signals. At 1004, the implantable medical device system determines, based on the environmental signals, one or more noises present in an ambient environment of a user of the implantable medical device system. At 1006, the implantable medical device system determines at least one user-preferred noise from the one or more noises. At 1008, the implantable medical device system attenuates the at least one user-preferred noise within the environmental signals to generate noise-reduced environmental signals. At 1010, the implantable medical device system generates, based on the noise-reduced environmental signals, one or more stimulation signals for delivery to the user of the implantable medical device system.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1-12. (canceled)

13. One or more non-transitory computer readable storage media comprising instructions that, when executed by a processor of a device configured to be implanted in or worn by a user, cause the processor to:
receive, at the device, noise model parameters from at least one external device in wireless communication with the device, wherein the noise model parameters represent noise detected by the at least one external device;
determine, based on sound signals received at the device and the noise model parameters, one or more noises present in an ambient environment of the device;
determine at least one user-preferred noise from the one or more noises for suppression;
suppress the at least one user-preferred noise within the sound signals to generate noise-suppressed sound signals; and
process the noise-suppressed sound signals for generation of stimulation signals for delivery to the user.

14. The one or more non-transitory computer readable storage media of claim 13, wherein the instructions executed to determine the one or more noises present in an ambient environment of the device include instructions that, when executed by the processor, cause the processor to:
reconstruct the noise detected by the at least one external device using the noise model parameters.

15. The one or more non-transitory computer readable storage media of claim 14, further comprising instructions that, when executed by the processor, cause the processor to:
filter the reconstructed noise with a user-specific profile to generate filtered reconstructed noise.

16. The one or more non-transitory computer readable storage media of claim 13, wherein the instructions executed to determine at least one user-preferred noise from the one or more noises for suppression include instructions that, when executed by the processor, cause the processor to:
provide a user of the device with an indication of the one or more noises present in an ambient environment; and
receive, from the user, a selection of the at least one user-preferred noise to suppress.

17. The one or more non-transitory computer readable storage media of claim 16, wherein the instructions executed to provide the user of the device with an indication of the one or more noises present in an ambient environment of the device comprise instructions that, when executed by the processor, cause the processor to:
display a list of the one or more noises present in an ambient environment.

18. The one or more non-transitory computer readable storage media of claim 17, wherein the instructions executed to display a list of the one or more noises present in an ambient environment include instructions that, when executed by the processor, cause the processor to:
classify noise in the ambient environment into one of a plurality of noise categories; and
display the plurality of noise categories to the user, wherein the user can select one of the plurality of noise categories for suppression.

19. The one or more non-transitory computer readable storage media of claim 13, wherein the instructions executed to determine at least one user-preferred noise from the one or more noises for suppression include instructions that, when executed by the processor, cause the processor to:
automatically determine the at least one user-preferred noise with a machine-learning prioritization module.

20. The one or more non-transitory computer readable storage media of claim 13, wherein the instructions executed to determine at least one user-preferred noise from the one or more noises for suppression include instructions that, when executed by the processor, cause the processor to:
determine at least one user-preferred noise source for suppression.

21. The one or more non-transitory computer readable storage media of claim 13, wherein the instructions executed to determine at least one user-preferred noise from the one or more noises for suppression include instructions that, when executed by the processor, cause the processor to:
determine at least one user-preferred noise type for suppression.

22. The one or more non-transitory computer readable storage media of claim 13, wherein the instructions executed to determine at least one user-preferred noise from the one or more noises for suppression include instructions that, when executed by the processor, cause the processor to:
classify noise in the ambient environment into one of a plurality of noise categories.

23. A method, comprising:
capturing environmental signals at device configured to be implanted in or worn by a user;
determining, based on the environmental signals, one or more noises present in an ambient environment of the user of the device;
determining at least one user-preferred noise from the one or more noises;
attenuating the at least one user-preferred noise within the environmental signals to generate noise-reduced environmental signals; and
generating, based on the noise-reduced environmental signals, one or more stimulation signals for delivery to the user.

24. The method of claim 23, wherein generating stimulation signals from noise-reduced environmental signals comprises:
generating electrical stimulation signals for delivery to the user.

25. The method of claim 23, wherein generating stimulation signals from the noise-reduced environmental signals comprises:
generating acoustic stimulation signals for delivery to the user.

26. The method of claim 23, wherein determining the at least one user-preferred noise from the one or more noises for suppression comprises:
providing the user with an indication of the one or more noises present in an ambient environment; and
receiving, from the user, a selection of the at least one user-preferred noise to suppress.

27. The method of claim 26, wherein providing the user of with an indication of the one or more noises present in an ambient environment of comprises:
displaying a list of the one or more noises present in an ambient environment.

28. The method of claim 27, wherein displaying the list of the one or more noises present in an ambient environment includes:
classifying noise in the ambient environment into one of a plurality of noise categories; and
displaying the plurality of noise categories to the user, wherein the user can select one of the plurality of noise categories for suppression.

29. The method of claim 23, wherein determining the at least one user-preferred noise from the one or more noises for suppression comprises:
automatically determining the at least one user-preferred noise with a machine-learning prioritization module.

30. The method of claim 23, wherein determining the at least one user-preferred noise from the one or more noises for suppression comprises:
determining at least one noise source for suppression.

31. The method of claim 23, wherein determining the at least one user-preferred noise from the one or more noises for suppression comprises:
determining at least one noise type for suppression.

32. The method of claim 23, wherein determining the at least one user-preferred noise from the one or more noises for suppression includes:
classifying noise in the ambient environment into one of a plurality of noise categories.

33. The method of claim 23, wherein capturing environmental signals comprises:
capturing environmental signals at one or more remote devices in wireless communication with the device.

34. The method of claim 33, wherein determining the at least one user-preferred noise from the one or more noises for suppression includes:
instructing the user to at least one of direct at least one of the one or more remote devices towards, or locate at least one of the one or more remote devices near, a noise source in the ambient environment.

35. The method of claim 23, wherein capturing environmental signals comprises:
capturing sound signals at the device.

36. The method of claim 23, wherein capturing environmental signals comprises:
capturing light signals at the device.

37. A system, comprising:
a user device is configured to be worn by a user comprising one or more sensors configured to capture environmental signals;
one or more remote devices in wireless communication with the user device, wherein the one or more remote devices each include at least one sensor configured to capture environmental signals; and
one or more processors configured to:
determine, based on the environmental signals, one or more noises present in an ambient environment of the user device,
determine at least one user-preferred noise from the one or more noises for suppression, and
suppress the at least one user-preferred noise within the environmental signals to generate noise-suppressed environmental signals.

38. The system of claim 37, wherein the environmental signals are sound signals.

39. The system of claim 37, wherein to determine at least one user-preferred noise from the one or more noises for suppression, the one or more processors are configured to:
provide the user of the user device with an indication of the one or more noises present in an ambient environment; and
receive, from the user, a selection of the at least one user-preferred noise to suppress.

40. The system of claim 37, wherein to determine at least one user-preferred noise from the one or more noises for suppression, the one or more processors are configured to:
automatically determine at least one user-preferred noise with a machine-learning prioritization module.
